(19) United States
(12) Patent Application Publication
(10) Pub. No.: US 2013/0035343 A1
(43) Pub. Date: Feb. 7, 2013

(54) COMBINATION OF ORGANIC COMPOUNDS

(71) Applicants: Diana Graus Porta, Basel (CH); Michael Shi, East Hanover, NJ (US); Dean Brent Evans, Therwil (CH)

(72) Inventors: Diana Graus Porta, Basel (CH); Michael Shi, East Hanover, NJ (US); Dean Brent Evans, Therwil (CH)

(73) Assignee: Novartis AG, Basel (CH)

(21) Appl. No.: 13/640,585

(22) PCT Filed: Apr. 14, 2011
(86) PCT No.: PCT/EP2011/055908

§ 371 Date: Oct. 11, 2012

Related U.S. Application Data
(60) Provisional application No. 61/325,018, filed on Apr. 16, 2010.

(51) Int. Cl. A61K 31/496; A61P 35/00

(52) U.S. Cl. 514/253.07

(57) ABSTRACT

A pharmaceutical combination comprising 4-Amino-5-fluoro-3-[6-(4-methylpiperazin-1-yl)-1H-benzimidazol-2-yl]-1H-quinolin-2-one and at least one mTOR inhibitor and the pharmaceutical combination for use in treating or preventing a proliferative disease.

The present invention relates to a pharmaceutical combination comprising 4-amino-5-fluoro-3-[6-(4-methylpiperazin-1-yl)-1H-benzimidazol-2-yl]-1H-quinolin-2-one or a pharmaceutically acceptable salt or a hydrate or a solvate and a mTOR inhibitor, and the uses of such a combination in the treatment of proliferative diseases, e.g. of a mTOR kinase dependent diseases.

In spite of numerous treatment options for proliferative disease patients, there remains a need for effective and safe anti-proliferative agents and a need for their preferential use in combination therapy.

It has now surprisingly been found that a combination comprising 4-amino-5-fluoro-3-[6-(4-methylpiperazin-1-yl)-1H-benzimidazol-2-yl]-1H-quinolin-2-one or a tautomer thereof, or a pharmaceutically acceptable salt or a hydrate or a solvate and at least one mTOR inhibitor, e.g. as defined below, has a beneficial effect on proliferative diseases, e.g. on mTOR kinase dependent diseases.

### 4-Amino-5-fluoro-3-[6-(4-methylpiperazin-1-yl)-1H-benzimidazol-2-yl]-1H-quinolin-2-one has the structure shown in Formula I

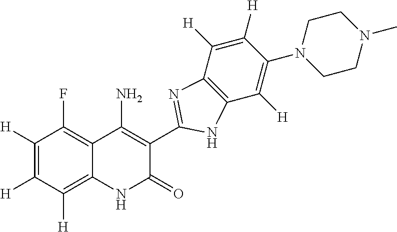 [SMILES: CN1CCN(c2ccc3nc(-c4c(N)c5c(F)cccc5[nH]c4=O)[nH]c3c2)CC1]

The compound of Formula I inhibits various protein kinases, such as tyrosine receptor kinases (RTKs). Consequently, the compound of Formula I and its salts are useful for inhibiting angiogenesis and treating proliferative diseases. Preparation of this compound and its salts, including the mono-lactic acid salt, are described in U.S. Pat. Nos. 6,605,617, 6,774,237, 7,335,774, and 7,470,709, and in U.S. patent application Ser. Nos. 10/982,757, 10/982,543, and 10/706,328, and in the published PCT applications WO 2006/127926 and WO2009/115562, each of which is incorporated herein by reference in its entirety.

The mono lactate salt of the compound of Formula I exists in a variety of polymorphs, including, e.g., the monohydrate form and the anhydrous form. Polymorphs occur where the same composition of matter (including its hydrates and solvates) crystallizes in a different lattice arrangement resulting in different thermodynamic and physical properties specific to the particular crystalline form.

Receptor tyrosine kinases (RTKs) are transmembrane polypeptides that regulate developmental cell growth and differentiation, remodeling and regeneration of adult tissues. Polypeptide ligands known as growth factors or cytokines, are known to activate RTKs. Signaling RTKs involves ligand binding and a shift in conformation in the external domain of the receptor resulting in its dimerization. Binding of the ligand to the RTK results in receptor trans-phosphorylation at specific tyrosine residues and subsequent activation of the catalytic domains for the phosphorylation of cytoplasmic substrates.

The compound of formula I inhibits tyrosine kinases. The tyrosine kinase is Cdc2 kinase (cell division cycle 2 kinase), Fyn (FYN oncogene kinase related to SRC, FGR, YES), Lck (lymphocyte-specific protein tyrosine kinase), c-Kit (stem cell factor receptor or mast cell growth factor receptor), p60src (tyrosine kinase originally identified as the v-src oncogene of the rous sarcoma viurs), c-ABL (tyrosine kinase that stands for an oncogene product originally isolated from the Adelson leukemia virus), VEGFR3, PDGFRα (platelet derived growth factor receptor α), PDGFRβ (platelet derived growth factor receptor β), FGFR3 (fibroblast growth factor receptor 3), FLT-3 (fms-like tyrosine kinase-3), or Tie-2 (tyrosine kinase with 1 g and EGF homology domains). In some embodiments, the tyrosine kinase is Cdc2 kinase, Fyn, Lck, or Tie-2 and in some other embodiments, the tyrosine kinase is c-Kit, c-ABL, p60src, VEGFR3, PDGFRα, PDGFRβ, FGFR3, or FLT-3.

Two subfamilies of RTKs are specific to the vascular endothelium. These include the vascular endothelial growth factor (VEGF) subfamily and the Tie receptor subfamily. Class III RTKs include vascular endothelial growth factor receptor 1 (VEGFR-1), vascular endothelial growth factor receptor 2 (VEGFR-2), and vascular endothelial growth factor receptor 3 (VEGFR-3).

The present technology relates to the use of 4-Amino-5-fluoro-3-[6-(4-methylpiperazin-1-yl)-1H-benzimidazol-2-yl]-1H-quinolin-2-one or a tautomer thereof, or a pharmaceutically acceptable salt or a hydrate or a solvate having the structure shown in Formula I:

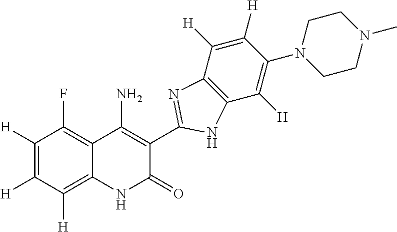 [SMILES: CN1CCN(c2ccc3nc(-c4c(N)c5c(F)cccc5[nH]c4=O)[nH]c3c2)CC1]

4-Amino-5-fluoro-3-[6-(4-methylpiperazin-1-yl)-1H-benzimidazol-2-yl]-1H-quinolin-2-one or a tautomer thereof, or a pharmaceutically acceptable salt can be administered at a dose of for example 500 mg per day, for example per os, for example in its lactate salt form thereof, for example in the monohydrate form of the monolactate salt thereof, for example 500 mg can be administered on a weekly basis as 5 days on treatment followed by two days off treatment.

Combinations of the invention include compounds which decrease or inhibit the activity/function of serine/threonine mTOR kinase. Such compounds will be referred to as “mTOR inhibitors” and include but is not limited to compounds, proteins or antibodies which inhibit members of the mTOR kinase family, e.g., RAD, rapamycin (sirolimus) and derivatives/analogs thereof such as everolimus or RAD001. Sirolimus is also known by the name RAPAMUNE and everolimus or RAD001 by the name CERTICAN or AFINITOR. Other compounds, proteins or antibodies which inhibit members of the mTOR kinase family include CCI-779, ABT578, SAR543, and ascomycin which is an ethyl analog of FK506. Also included are AP23573 and AP23841 from Ariad.

Suitable mTOR inhibitors include e.g.:
I. Rapamycin which is an immunosuppressive lactam macrolide that is produced by Streptomyces hygroscopicus.
II. Rapamycin derivatives such as:
   a. substituted rapamycin e.g. a 40-O-substituted rapamycin e.g. as described in U.S. Pat. No. 5,258,389, WO 94/09010, WO 92/05179, U.S. Pat. No. 5,118,677, U.S. Pat. No. 5,118,678, U.S. Pat. No. 5,100,883, U.S. Pat. No. 5,151,413, U.S. Pat. No. 5,120,842, WO 93/11130, WO 94/02136, WO 94/02485 and WO 95/14023 all of which are incorporated herein by reference;
   b. a 16-O-substituted rapamycin e.g. as disclosed in WO 94/02136, WO 95/16691 and WO 96/41807, the contents of which are incorporated herein by reference;
   c. a 32-hydrogenated rapamycin e.g. as described in WO 96/41807 and U.S. Pat. No. 5,256,790, incorporated herein by reference.
   d. rapamycin derivatives which are compounds of formula II

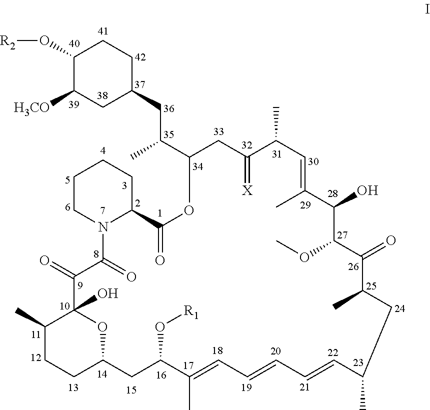

wherein
R₁ is CH₃ or C₃₋₆alkynyl,
R₂ is H or —CH₂—CH₂—OH, 3-hydroxy-2-(hydroxymethyl)-2-methyl-propanoyl or tetrazolyl, and X is ═O, (H,H) or (H,OH)
provided that R₂ is other than H when X is ═O and R₁ is CH₃,
or a prodrug thereof when R₂ is —CH₂—CH₂—OH, e.g. a physiologically hydrolysable ether thereof.

Compounds of formula I are disclosed e.g. in WO 94/09010, WO 95/16691 or WO 96/41807, which are incorporated herein by reference. They may be prepared as disclosed or by analogy to the procedures described in these references.

Compounds may be 32-deoxorapamycin, 16-pent-2-ynyloxy-32-deoxorapamycin, 16-pent-2-ynyloxy-32(S)-dihydro-rapamycin, 16-pent-2-ynyloxy-32(S)-dihydro-40-O-(2-hydroxyethyl)-rapamycin and, 40-0-(2-hydroxyethyl)-rapamycin, disclosed as Example 8 in WO 94/09010.

Rapamycin derivatives may be of formula I are 40-O-(2-hydroxyethyl)-rapamycin, 40-[3-hydroxy-2-(hydroxymethyl)-2-methylpropanoate]-rapamycin (also called CCI779), 40-epi-(tetrazolyl)-rapamycin (also called ABT578), 32-deoxorapamycin, 16-pent-2-ynyloxy-32(S)-dihydro rapamycin, or TAFA-93.
e. Rapamycin derivatives also include so-called rapalogs, e.g. as disclosed in WO 98/02441 and WO 01/14387, e.g. AP23573, AP23464, or AP23841.

Rapamycin and derivatives thereof have, on the basis of observed activity, e.g. binding to macrophilin-12 (also known as FK-506 binding protein or FKBP-12), e.g. as described in WO 94/09010, WO 95/16691 or WO 96/41807, been found to be useful e.g. as immunosuppressant, e.g. in the treatment of acute allograft rejection.

Ascomycin, which is an ethyl analog of FK506.

AZD08055 and OSI127, which are compounds that inhibit the kinase activity of mTOR by directly binding to the ATP-binding cleft of the enzyme.

A preferred mTOR inhibitor is 40-O-(2-hydroxy)ethyl-rapamycin (everolimus).

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the technology can be prepared and administered as described in the cited documents, respectively. Also within the scope of this invention is the combination of more than two separate active ingredients as set forth above, i.e., a pharmaceutical combination within the scope of this invention could include three active ingredients or more.

Provided is a pharmaceutical combination comprising:

a) 4-Amino-5-fluoro-3-[6-(4-methylpiperazin-1-yl)-1H-benzimidazol-2-yl]-1H-quinolin-2-one has the structure shown in Formula I:

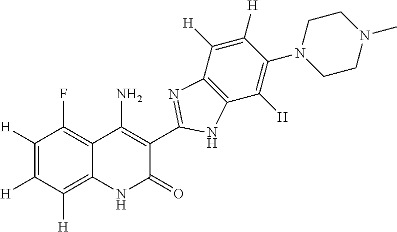 [SMILES: CN1CCN(c2ccc3nc(-c4c(N)c5c(F)cccc5[nH]c4=O)[nH]c3c2)CC1]

and
b) at least one mTOR inhibitor.

In another aspect the use of 4-Amino-5-fluoro-3-[6-(4-methylpiperazin-1-yl)-1H-benzimidazol-2-yl]-1H-quinolin-2-one and at least one mTOR inhibitor for the manufacture of a medicament for the treatment or prevention of a proliferative disease is provided. 4-Amino-5-fluoro-3-[6-(4-methylpiperazin-1-yl)-1H-benzimidazol-2-yl]-1H-quinolin-2-one and at least one mTOR inhibitor may be administered separately, simultaneously or sequentially.

In a further aspect the use of 4-Amino-5-fluoro-3-[6-(4-methylpiperazin-1-yl)-1H-benzimidazol-2-yl]-1H-quinolin-2-one and at least one mTOR inhibitor for the manufacture of a medicament for the treatment or prevention of a (mTOR) kinase dependent disease is provided. 4-Amino-5-fluoro-3-[6-(4-methylpiperazin-1-yl)-1H-benzimidazol-2-yl]-1H-quinolin-2-one and at least one mTOR inhibitor may be administered separately, simultaneously or sequentially.

In another aspect the invention pertains to a combination of
1) 4-Amino-5-fluoro-3-[6-(4-methylpiperazin-1-yl)-1H-benzimidazol-2-yl]-1H-quinolin-2-one or a tautomer thereof, or a pharmaceutically acceptable salt or a hydrate or a solvate, e.g. the lactate salt of 4-Amino-5-fluoro-3-[6-(4-methylpiperazin-1-yl)-1H-benzimidazol-2-yl]-1H-quinolin-2-one and
2) at least one mTOR inhibitor, e.g. a suitable mTOR inhibitor as described above, for example everolimus,
for use in treating or preventing a proliferative disease, or preventing the progression of a proliferative disease or of a (mTOR) dependent disease, e.g. breast cancer, bladder cancer, urothelial cancer, gastrointestinal cancer, neuroendocrine tumors, lymphomas, hepatocellular carcinoma or liver cancer and prostate cancer, carcinoma of the brain, kidney, e.g. renal cell carcinoma (RCC), adrenal gland cancer, stomach cancer, cancer of the ovary, pancreas cancer, lung cancer, vagina or thyroid, sarcoma, glioblastomas, multiple myeloma or colon carcinoma or colorectal adenoma or a tumor of the neck and head, an epidermal hyperproliferation, psoriasis, prostate hyperplasia, a neoplasia, a neoplasia of epithelial character, adenoid cystic carcinoma (ACC), hepatocellular carcinoma (HCC) or a leukemia.

The present invention also pertains to a combination of
1) 4-Amino-5-fluoro-3-[6-(4-methylpiperazin-1-yl)-1H-benzimidazol-2-yl]-1H-quinolin-2-one or a tautomer thereof, or a pharmaceutically acceptable salt or a hydrate or a solvate, e.g. the lactate salt of 4-Amino-5-fluoro-3-[6-(4-methylpiperazin-1-yl)-1H-benzimidazol-2-yl]-1H-quinolin-2-one and
2) everolimus,
for use in treating or preventing, or preventing the progression of a disease selected from breast cancer, bladder cancer, urothelial cancer, gastrointestinal cancer, neuroendocrine tumors, lymphomas, multiple myeloma, hepatocellular carcinoma or liver cancer and prostate cancer, kidney, e.g. renal cell carcinoma (RCC), adenoid cystic carcinoma (ACC), hepatocellular carcinoma (HCC).
4-Amino-5-fluoro-3-[6-(4-methylpiperazin-1-yl)-1H-benzimidazol-2-yl]-1H-quinolin-2-one and at least one mTOR inhibitor may be administered separately, simultaneously or sequentially.

In some embodiments a method of treating or preventing a disease by administering a compound of 4-Amino-5-fluoro-3-[6-(4-methylpiperazin-1-yl)-1H-benzimidazol-2-yl]-1H-quinolin-2-one and at least one mTOR inhibitor is provided. The disease to be treated may be a proliferative disease or a mTOR dependent disease. 4-Amino-5-fluoro-3-[6-(4-methylpiperazin-1-yl)-1H-benzimidazol-2-yl]-1H-quinolin-2-one and at least one mTOR inhibitor may be administered separately, simultaneously or sequentially.

The mTOR inhibitor may be selected from RAD rapamycin (sirolimus) and derivatives/analogs thereof such as everolimus or RAD001; CCI-779, ABT578, SAR543, ascomycin (an ethyl analog of FK506), AP23573, AP23841, AZD08055 and OSI027.

A preferred mTOR inhibitor is 40-O-(2-hydroxy)ethyl-rapamycin (everolimus). Everolimus can be administered as follows: at least 2.5 mg/day or 5 to 10 mg/day, e.g. 10 mg/day.

The term “mTOR kinase dependent diseases” includes but is not restricted to the following diseases and conditions:
Organ or tissue transplant rejection, e.g. for the treatment of recipients of e.g. heart, lung, combined heart-lung, liver, kidney, pancreatic, skin or corneal transplants; graft-versus-host disease, such as following bone marrow transplantation;
Restenosis
Hamartoma syndromes, such as tuberous sclerosis or Cowden Disease
Lymphangioleiomyomatosis
Retinitis pigmentosis
Autoimmune diseases including encephalomyelitis, insulin-dependent diabetes mellitus, lupus, dermatomyositis, arthritis and rheumatic diseases
Steroid-resistant acute Lymphoblastic Leukaemia
Fibrotic diseases including scleroderma, pulmonary fibrosis, renal fibrosis, cystic fibrosis
Pulmonary hypertension
Immunomodulation
Multiple sclerosis
VHL syndrome
Carney complex
Familial adenonamtous polyposis
Juvenile polyposis syndrome
Birt-Hogg-Duke syndrome
Familial hypertrophic cardiomyopathy
Wolf-Parkinson-White syndrome
Neurodegenarative disorders such as Parkinson's, Huntingtin's, Alzheimer's and dementias caused by tau mutations, spinocerebellar ataxia type 3, motor neuron disease caused by SODI mutations, neuronal ceroid lipofucinoses/Batten disease (pediatric neurodegeneration)
wet and dry macular degeneration
muscle wasting (atrophy, cachexia) and myopathies such as Danon's disease.
bacterial and viral infections including M. tuberculosis, group A streptococcus, HSV type I, HIV infection
Neurofibromatosis including Neurofibromatosis type 1,
Peutz-Jeghers syndrome

Furthermore, “mTOR kinase dependent diseases” include cancers and other related malignancies. A non-limiting list of the cancers associated with pathological mTOR signaling cascades includes breast cancer, renal cell carcinoma, urothelial cancer, gastric tumors, neuroendocrine tumors, lymphomas, multiple myeloma, adenoid cystic carcinoma, hepatocellular and prostate cancer.

Examples for a proliferative disease are for instance benign or malignant tumor, carcinoma of the brain, kidney, e.g. renal cell carcinoma (RCC), liver, adrenal gland, bladder, breast, stomach, urothelial carcinoma, gastric tumors, ovaries, colon, rectum, prostate, pancreas, lung, vagina or thyroid, sarcoma, glioblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, lymphomas, adenoid cystic carcinoma, a mammary carcinoma, hepatocellular carcinoma (HCC) or a leukemia.

Suitable clinical studies may be, for example, open label, dose escalation studies in patients with proliferative diseases. Such studies prove in particular the synergism of the active ingredients of the combination of the invention. The beneficial effects on proliferative diseases may be determined directly through the results of these studies which are known as such to a person skilled in the art. Such studies may be, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) may be administered in a fixed dose and the dose of agent (b) may be escalated. Each patient may receive doses of the agent (a) either daily or intermittent. The efficacy of the treatment may be determined in such studies, e.g., after 12, 18 or 24 weeks by evaluation of symptom scores every 6 weeks.

The administration of a pharmaceutical combination of the invention may result not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit may be that lower doses of the active ingredients of the combination of the invention may be used, for example, that the dosages need not only often be smaller but may also be applied less frequently, which may diminish the incidence or severity of side-effects. This is in accordance with the desires and requirements of the patients to be treated.

Provided is a pharmaceutical composition comprising a quantity, which may be jointly therapeutically effective at treating or preventing proliferative diseases with the combination. In this composition, agent (a) and agent (b) may be administered together, one after the other or separately in one combined unit dosage form or in two separate unit dosage forms. The unit dosage form may also be a fixed combination.

The pharmaceutical compositions for separate administration of agent (a) and agent (b) or for the administration in a fixed combination, i.e. a single galenical composition comprising at least two combination partners (a) and (b), according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g. as indicated above, or in combination with one or more pharmaceutically acceptable carriers or diluents, especially suitable for enteral or parenteral application.

Suitable pharmaceutical compositions may contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount may be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating proliferative diseases may comprise (i) administration of the first agent (a) in free or pharmaceutically acceptable salt form and (ii) administration of an agent (b) in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to the conditions being treated.

Short description of the Figures:

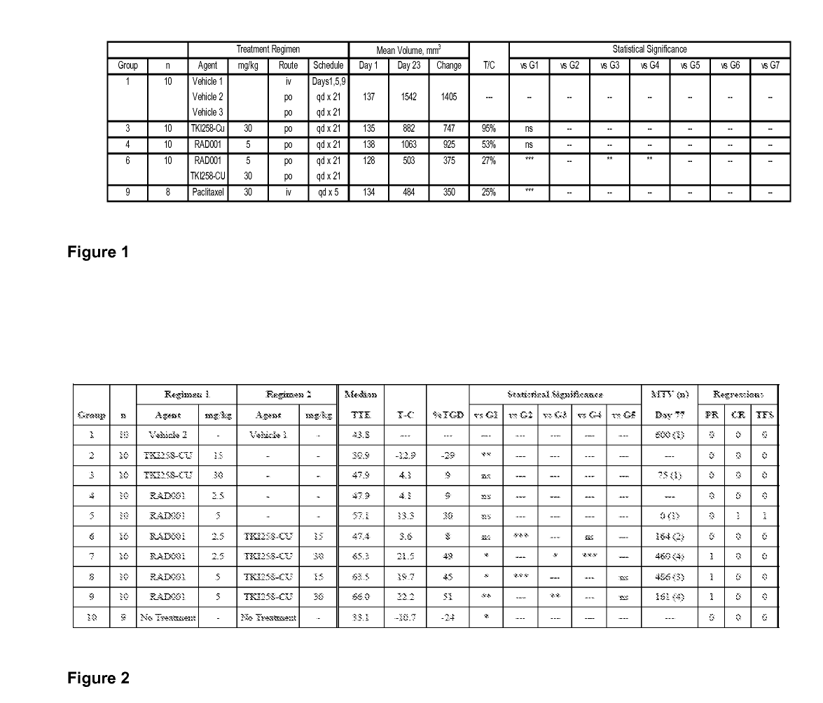

FIG. 1/6 shows the tumor growth of a Caki-1 tumor line derived from a human renal clear cell carcinoma in nude mice up to Day 23 for Groups 1, 3, 4, 6 and 9 when treated with Compound of Formula I, RAD001 and the combination of both.

FIG. 2/6 shows the tumor growth of a 786-O tumor line from a human primary clear cell renal carcinoma in nude mice up to Day 77 for Groups 1 to 10 when treated with Compound of Formula I, RAD001 and the combination of both.

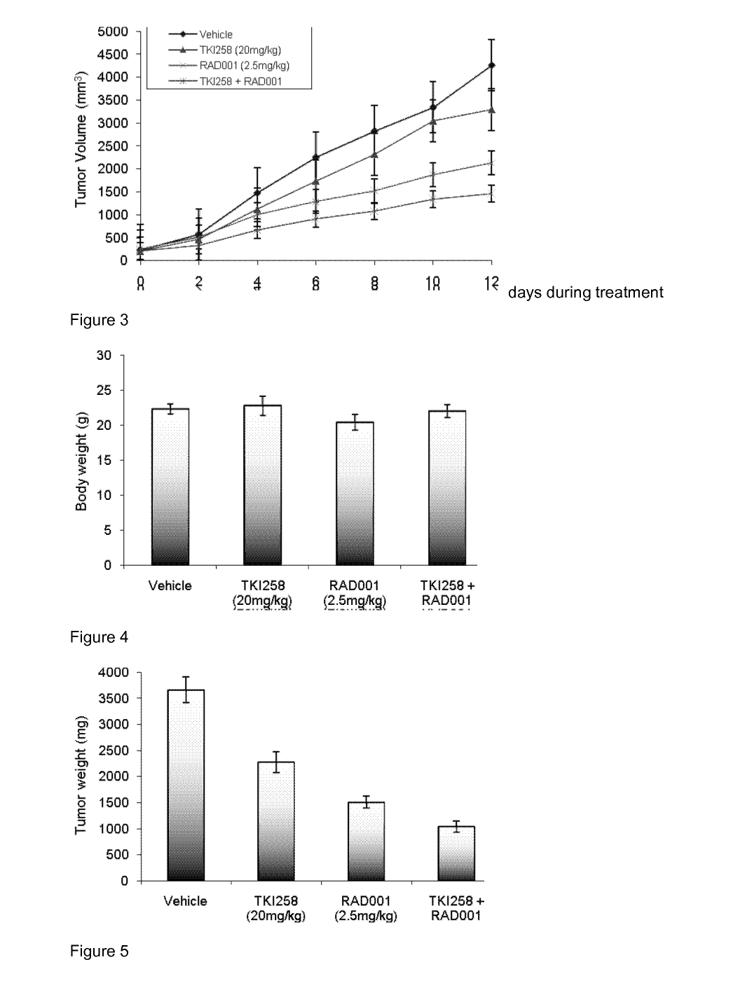

FIG. 3/6 shows the tumor volume (tumor growth) when animals were treated with Compound of Formula I, RAD001 and the combination of both over time.

FIG. 4/6 shows the average body weight of the animals with vehicle, Compound of Formula I, RAD001 or combination treatment.

FIG. 5/6 shows tumor weight when animals were treated with vehicle, Compound of Formula I, RAD001 or the combination.

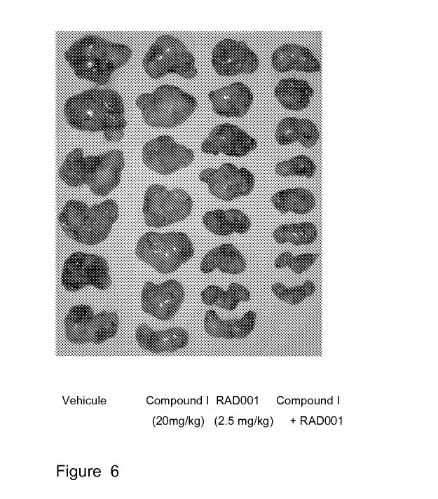

FIG. 6/6 shows pictures of tumors when animals were treated with vehicle, Compound of Formula I, RAD001 or the combination.

Following is a description by way of examples.

### EXAMPLE 1

The Caki-1 tumor line is derived from a skin metastasis of a human renal clear cell carcinoma. The tumors are maintained by engraftment in nude mice. A 1 mm³ fragment is implanted subcutaneously in the right flank of each test animal. The tumors are measured with calipers twice weekly, and daily as the mean volume approached 100-150 mm³. Fifteen days after tumor cell implantation, on D1 (day 1) of the study, the animals are sorted into nine groups of ten mice, with individual tumor sizes of 75-196 mm³ and group mean tumor sizes of 128-138 mm³. Tumor size, in mm³, is calculated from

$$Tumor volume = \frac{{w}^{1} \times 1}{2}$$

where “w” is the width and “1” is the length, in mm, of the tumor. Tumor weight is estimated with the assumption that 1 mg is equivalent to 1 mm³ of tumor volume.

For the efficacy study RAD001 and its vehicle (Vehicle 2) and TKI258-CU and its vehicle (Vehicle 3) are each administered orally (p.o.), once daily for twenty-one consecutive days (qd×21). Paclitaxel is administered i.v., once daily on alternate days for five doses (qod×5). All drugs in combination are administered within 30-60 minutes. The dosing volume, 10 mL/kg (0.2 mL/20 g mouse), is scaled to the weight of each animal as determined on the day of dosing, except on weekends when the previous BW was carried forward.

Groups of nude mice (n=10/group) are treated as follows. Group 1 mice receives

the RAD001 vehicle (Vehicle 2), and the TKI258-CU vehicle (Vehicle 3), and serves as controls for all analyses. Additionally group one receives a vehicle (Vehicle 1) for another drug which is not part of this application. Group 3 receives TKI258-CU monotherapy at 30 mg/kg (equivalent to 23.5 mg/kg free base). Group 4 receives RAD001 monotherapy at 5 mg/kg. Group 6 receives 5 mg/kg RAD001 in dual combination with 30 mg/kg TKI258-CU.

Group 9 mice receives 30 mg/kg paclitaxel as a positive reference therapy.

The study begins on Day 1 (D1). Efficacy is determined from tumor volume changes up to D23 (day 23). Efficacy is determined on D23.

For the purpose of statistical and graphical analyses, ΔTV, the difference in tumor volume between D1 (the start of dosing) and the endpoint day, was determined for each animal. For each treatment group, the response on the endpoint day was calculated by the following relation:

T/C(%)=100×ΔT/ΔC, for ΔT>0

Where ΔT=(mean tumor volume of the drug-treated group on the endpoint day)−(mean tumor volume of the drug-treated group on D1), and ΔC=(mean tumor volume of the control group on the endpoint day)−(mean tumor volume of the control group on D1).

A treatment that achieved a T/C value of 40% or less was classified as potentially therapeutically active.

FIG./Table 1/2 shows the treatment response up to Day 23. (n) is the number of animals in a group not dead from treatment-related, accidental, or unknown causes. The Mean Volume is the group mean tumor volume; The Change is the difference between D1 and D23. T/C is 100×(ΔT/ΔC) which is the percent change between Day 1 and Day 23 in the mean tumor volume of treated group (ΔT) compared with change in control Group 1 (ΔV). Statistical significance is shown by Kruskal-Wallis with post hoc Dunn's multiple comparison test): ns=not significant; *=p<0.05; **=p<0.01; and ***=p<0.0001, compared to the indicated group (G1 to G7).

In Group 6 (FIG./Table 1/2), dual therapy with 5 mg/kg RAD001 and 30 mg/kg TKI258-CU resulted in a ΔT of 375 mm3, corresponding to 27% T/C, and produced significant median growth inhibition (P<0.001). The dual therapy provided significant (P<0.01) improvements over TKI258-CU and RAD001 mono therapies in Groups 3 and 4, respectively.

### EXAMPLE 2

The 786-O tumor line is derived from a human primary clear cell renal carcinoma. The tumors are maintained by engraftment in nude mice. 0.2 ml of 786-O cell suspension (1×10⁷ cells) are inoculated subcutaneously in the right flank of each nude mouse. The tumors are caliprated twice weekly, and daily as the mean volume approached 150-220 mm³. Eight days after tumor cell implantation, on D1 (day 1) of the study, the animals are sorted into ten groups of ten mice, with individual tumor sizes of 172-196 mm³ and group mean tumor sizes of 174 mm³. Tumor size, in mm³, is calculated from

$$Tumor volume = \frac{{w}^{1} \times 1}{2}$$

where “w” is the width and “1” is the length, in mm, of the tumor. Tumor weight is estimated with the assumption that 1 mg is equivalent to 1 mm³ of tumor volume.

For the efficacy study all treatments (TKI258 and RAD001) were administered by oral gavage (p.o.) once daily for twenty-one consecutive days (qd×21). For combination therapies, TKI258 is given 60 minutes after RAD001. The dosing volume, 10 mL/kg (0.2 mL/20 g mouse), is scaled to the weight of each animal as determined on the day of dosing, except on weekends when the previous BW was carried forward.

10 Groups of nude mice (n=10/group) are treated as follows. Group 1 mice receive both vehicles, and serve as controls for all analyses. Group 10 mice are not treated, and serve as controls for the vehicle treatments. Group 2 and 3 receive TKI258-CU mono therapies at 15 and 30 mg/kg (doses equivalent to 11.7 and 23.4 mg/kg of free base), respectively. Groups 4 and 5 receive RAD001 mono therapies at 2.5 and 5 mg/kg, respectively. Groups 6 and 7 receive 2.5 mg/kg RAD001 in combination with 15 and mg/kg TKI258-CU, respectively. Groups 8 and 9 receive 5 mg/kg RAD001 in combination with 15 and 30 mg/kg TKI258-CU, respectively.

The study begins on Day 1 (D1). Long term efficacy is determined from tumor volume changes up to D77 (day 77) or to the endpoint volume of the tumor (800 mm³)

For the purpose of statistical and graphical analyses, ΔTV, the difference in tumor volume between D1 (the start of dosing) and the endpoint day, is determined for each animal. For each treatment group, the response on the endpoint day was calculated by the following relation:

T/C(%)=100×ΔT/ΔC, for ΔT>0

where ΔT=(mean tumor volume of the drug-treated group on the endpoint day)−(mean tumor volume of the drug-treated group on D1), and ΔC=(mean tumor volume of the control group on the endpoint day)−(mean tumor volume of the control group on D1).

A treatment that achieved a T/C value of 40% or less was classified as potentially therapeutically active.

Each animal was euthanized when its neoplasm reached the endpoint volume (800 mm3),

or on the last day of the study (D77). For each animal whose tumor reached the endpoint
volume, the time to endpoint (TTE) was calculated by the following equation:

$$T T E = \frac{log 10 ( endpoint volume ) - b}{m}$$

where TTE is expressed in days, endpoint volume is in mm3, b is the intercept, and m is the slope of the line obtained by linear regression of a log-transformed tumor growth data set. The data set is comprised of the first observation that exceeded the study endpoint volume and the three consecutive observations that immediately preceded the attainment of the endpoint volume. The calculated TTE is usually less than the day on which an animal is euthanized for tumor size. An animal with a tumor that did not reach the endpoint is assigned a TTE value equal to the last day. An animal classified as having died from treatment-related (TR) causes or non-treatment-related metastasis (NTRm) is assigned a TTE value equal to the day of death. An animal classified as having died from non-treatment-related (NTR) causes is excluded from TTE calculations.

Treatment efficacy was determined from tumor growth delay (TGD), which is defined as the increase in the median TTE for a treatment group compared to the control group:

TGD=T−C, expressed in days, or as a percentage of the median TTE of the control group:

$$\% T G D = \frac{T - C}{C} \times 100$$

where T is the median TTE for a treatment group and C is TTE for control group 1.

Treatment efficacy may also be determined from the tumor volumes of animals remaining in the study on the last day, and from the number of regression responses. The MTV(n) is defined as the median tumor volume on D77 in the number of animals remaining, n, whose tumors had not attained the endpoint volume.

Treatment may cause partial regression (PR) or a complete regression (CR) of the tumor in a animal. A PR indicates that the tumor volume is 50% or less of its D1 volume for three consecutive measurements during the course of the study, and equal to or greater than 13.5 mm3 for one or more of these three measurements. A CR indicates that the tumor volume was less than 13.5 mm3 for three consecutive measurements during the course of the study. An animal with a CR at the termination of a study is additionally classified as a tumor-free survivor (TFS).

FIG./Table 2/2 shows the treatment response up to the study endpoint (D77, day 77 or tumor volume of 800 mm³ which ever comes first), (n) is the number of animals in a group not dead from treatment-related, accidental, or unknown causes. TTE is the time to endpoint; T−C is the difference between median TTE (days) of treated versus control group; % TGD=[(T−C)/C]×100. The statistical significance is analysed by the Logrank test: ns=not significant; *=p<0.05; **=p<0.01; and ***=p<0.0001, compared to the indicated group (G1 to G5). MTV (n) is the median tumor volume (mm³) for the number of animals on the day of TGD analysis (excludes animals with tumor volume at endpoint).

### Efficacy of the 77 Days Study

In Group 7, combination of 2.5 mg/kg RAD001 with 30 mg/kg TKI258-CU resulted in a median TTE of 65.3 days, corresponding to a % TGD of 49. The survival extension was significant (P<0.05). The combination significantly improved upon the corresponding TKI258-CU mono therapy in Group 3 (P<0.05) and the corresponding RAD001 Mono therapy in Group 4 (P<0.001). Four Group 7 animals survived to D77 with an MTV of 460 mm3, and one PR response occurred.

In Group 8, combination of 5 mg/kg RAD001 with 15 mg/kg TKI258-CU resulted in a median TTE of 63.5 days, corresponding to a % TGD of 45. The survival extension was significant (P<0.05). The combination significantly improved upon the corresponding TKI258-CU mono therapy in Group 2 (P<0.001), and non-significantly upon the corresponding RAD001 mono therapy in Group 5. Three Group 8 animals survived to D77 with an MTV of 486 mm3, and one PR response occurred.

In Group 9, combination of 5 mg/kg RAD001 with 30 mg/kg TKI258-CU resulted in a median TTE of 66.0 days, corresponding to a % TGD of 51. The survival extension was significant (P<0.01). The combination significantly improved upon the corresponding TKI258-CU mono therapy in Group 3 (P<0.01), and non-significantly upon the corresponding RAD001 mono therapy in Group 5. Four Group 9 animals survived to D77 with an MTV of 161 mm3, and one PR response occurred.

### EXAMPLE 3

Xenograft models: All mice were provided with sterilized food and water ad libitum and housed in negative pressure isolators with 12 hours light/dark cycle. Primary HCCs have previously been used to create xenograft lines, of which the following lines (07-0409, 29-0909A, 01-0909) were used to establish tumors in male SCID mice (Animal Resources Centre, Canning Vale, Western Australia) aged 9 to 10 weeks.

Tumor treatment: Compound of Formula I and RAD001 was dissolved in vehicle at an appropriate concentration before treatment. Mice bearing indicated tumors were orally administered 5 mg/kg RAD001 or 30 mg/kg Compound of Formula I daily, or two compounds combined for indicated days. Each treatment group was comprised of 10 animals and each experiment was repeated at least twice. Treatment started on day 7 after tumor implantation. By this time, the tumors reached the size of approximately 100 mm³. Tumor growth was monitored and tumor volume was calculated as described. At the end of the study, the mice were sacrificed with body and tumor weights recorded and the tumors harvested for analysis. The efficacy of Compound of Formula I was determined by T/C ratio, where T and C are median weight of drug-treated and vehicle-treated tumor respectively at the end of treatment. T/C ratios less than 0.42 are considered active as determined according to the criteria of Drug Evaluation Branch of the Division of Cancer Treatment, National Cancer Institute.

Results: The anti-tumor activities of Compound of Formula I on patient-derived HCC xenograft lines (07-0409, 29-0909A, 01-0909) were observed, data shown only for HCC07-0409. Throughout the course of treatment, no significant weight loss and no acute mortality were observed indicating that Compound of Formula I treatment was safe and of acceptable toxicity. FIGS. 3/6, 5/6 and 6/6 showed that the tumor growth rate of xenografts was inhibited by Compound of Formula I or RAD001 single agent therapy, but did not induce tumor regressions. When two agents were combined, the antitumor effect was significantly better than single agent alone, indicating synergistic effect of the two compounds.

1. A pharmaceutical combination comprising
a) 4-Amino-5-fluoro-3-[6-(4-methylpiperazin-1-yl)-1H-benzimidazol-2-yl]-1H-quinolin-2-one or a tautomer thereof or a mixture thereof, or a pharmaceutically acceptable salt thereof,
b) at least one mTOR inhibitor.

2. A pharmaceutical combination according to claim 1 wherein the mTOR inhibitor is selected from RAD rapamycin (sirolimus) and derivatives/analogs thereof such as everolimus or RAD001; CCI-779, ABT578, SAR543, ascomycin (an ethyl analog of FK506), AP23573, AP23841, AZD08055 and OSI027.

3. A pharmaceutical combination according to claim 1 wherein the mTOR inhibitor is everolimus.

4. (canceled)

5. The method according to claim 10 wherein the mTOR inhibitor is selected from RAD rapamycin (sirolimus) and derivatives/analogs thereof such as everolimus or RAD001; CCI-779, ABT578, SAR543, ascomycin (an ethyl analog of FK506), AP23573, AP23841, AZD08055 and OSI027.

6. The method according to claim 5 wherein the mTOR inhibitor is everolimus.

7. (canceled)

8. A pharmaceutical combination according to claim 1 for use in treating or preventing a proliferative disease or a (mTOR) kinase dependent disease.

9. The combination according to claim 1 wherein 4-Amino-5-fluoro-3-[6-(4-methylpiperazin-1-yl)-1H-benzimidazol-2-yl]-1H-quinolin-2-one and at least one mTOR inhibitor are administered separately, simultaneously or sequentially.

10. A method of treating or preventing a proliferative disease or a mTOR kinase dependent disease by administering the combination of claim 1.

11. The method according to claim 10 wherein 4-Amino-5-fluoro-3-[6-(4-methylpiperazin-1-yl)-1H-benzimidazol-2-yl]-1H-quinolin-2-one and at least one mTOR inhibitor are administered separately, simultaneously or sequentially.

12. Combination according to claim 1 to wherein 4-Amino-5-fluoro-3-[6-(4-methylpiperazin-1-yl)-1H-benzimidazol-2-yl]-1H-quinolin-2-one one or a tautomer thereof or a mixture thereof, or a pharmaceutically acceptable salt thereof is administered at the dose of 500 mg per day, 5 days on/2 days off.

13. A pharmaceutical combination according to claim 2 wherein everolimus is administered at the dose of at least 2.6 mg/day.

14. A pharmaceutical combination according to claim 13 where everolimus is administered at a dose of 5 to 10 mg/day.

15. A pharmaceutical combination according to claim 1 wherein 4-Amino-5-fluoro-3-[6-(4-methylpiperazin-1-yl)-1H-benzimidazol-2-yl]-1H-quinolin-2-one or a tautomer thereof or a mixture thereof is in its lactate salt form thereof.

16. A pharmaceutical combination according to claim 1 for use in the treatment or prevention of progression of a disease selected from: breast cancer, neuroendocrine tumors, lymphomas, hepatocellular carcinoma, renal cell carcinoma, multiple myeloma, urothelial carcinoma, bladder cancer, endometrial cancer, brain carcinoma and endometrial carcinoma.

17. The method according to claim 10 wherein the proliferative disease is selected from breast cancer, neuroendocrine tumors, lymphomas, hepatocellular carcinoma, renal cell carcinoma, multiple myeloma, urothelial carcinoma, bladder cancer, endometrial cancer, brain carcinoma and endometrial carcinoma.
